(19) United States
(12) Patent Application Publication
(10) Pub. No.: US 2002/0123442 A1
(43) Pub. Date: Sep. 5, 2002

(54) Dual function bar soap

(76) Inventor: Audrey Pawlikowski, Clinton, NJ (US)

Correspondence Address: Audrey Pawlikowski, 39 Overlook Drive, Clinton, NJ, 08809, US

(21) Appl. No.: 10/078,583

(22) Filed: Feb. 20, 2002

Related U.S. Application Data
(60) Provisional application No. 60/272,880, filed on Mar. 5, 2001.

(51) Int. Cl. A61K 7/50

(52) U.S. Cl. 510/141; 510/146; 510/148

(57) ABSTRACT

The present invention is a dual function bar formed into a single structure, with (a.) a first portion having at least one exposed surface and containing a deodorant soap formula; and, (b.) a second portion distinct from the first portion and having at least one exposed surface and containing a moisturizing soap formula. The soap may have a third portion, being a neutral portion absent deodorant and moisturizing formulae, and typically being centrally located and acting as a substrate or support for the other portions. For example, it may be one or more layers, or it may contain recesses to receive the other portions in appropriate areas, such as on opposite sides or opposite ends.

### REFERENCE TO RELATED CASES

This application is based on U.S. pending provisional application Serial No. 60/272,880 filed on Mar. 5, 2001 by the inventor herein.

### BACKGROUND OF THE INVENTION

1. Field of the Invention

The present invention is directed to a dual function bar of soap which offers a deodorant-based soap formulation on one portion and a beauty-based soap formulation on another portion, typically on opposite sides thereof. This enables a user to wash one body area with a deodorant soap and another body area with a beauty soap, without switching soaps, and also eliminating different clashing scents left on the skin.

2. Information Disclosure Statement

Soap having moisturizing formulae are well known. Likewise soaps having deodorizing formulae are well known. The following patents are representative of the types of actives in the art of soap making:

U.S. Pat. No. 4,288,341 descirbes a deodorant toilet bar comprises a soap mixture; optionally, straight chain fatty acids; and a deodorant composition. The bar can be employed for personal washing of the skin and will as a result reduce human body malodor.

U.S. Pat. No. 5,547,602 describes soap bars which both clean and moisturize the skin are disclosed. The soap bars contain, in addition to conventional bar soap components, a mixture of petrolatum and a specifically defined emollient, preferably an hydroxylated milk glyceride.

U.S. Pat. No. 5,573,699 describes deodorant soap or detergent compositions comprising a surfacant, e.g., a soap or synthetic detergent, and as a deodorizing component, a minor amount of a sodium zinc cabonate hydrate complex salt. The letter salt may be prepared by reacting zinc oxide and sodium bicarbonate in the presence of water.

U.S. Pat. No. 5,691,287 describes a cleansing bar that includes (a) from about 20% to about 35%, by weight of all ingredients in the cleansing bar, a synthetic detergent, sodium cocoyl isethionate, (b) from about 6% to about 11%, by weight of all ingredients in the cleansing bar, cetyl alcohol, wherein a mole to mole ratio of (a) to (b) is at least about 2:2:1, (c) at least about 30%, by weight of all ingredients in the cleansing bar, processed grain, (d) a buffer for adjusting the pH of the cleansing bar to be in range from about 4.0 to about 5.5, and (e) from about 9% to about 20%, by weight of all ingredients in the cleansing bar, water. Also disclosed are methods of making such a cleansing bar.

Notwithstanding the prior art, the present invention is neither taught nor rendered obvious thereby.

### SUMMARY OF THE INVENTION

The present invention is directed to a dual function bar soap. It is essentially a bar soap formed into a single structure, which includes (a.) a first portion having at least one exposed surface and containing a deodorant soap formula; and, (b.) a second portion distinct from said first portion and having at least one exposed surface and containing a moisturizing soap formula. The soap of the present invention may be an elongated bar soap having a top and a bottom and a first end and a second end, such that one of said first portion and said second portion is the top and the other of said first portion and said second portion is the bottom, or, one of said first portion and said second portion is the first end and the other of said first portion and said second portion is the second end.

In some preferred embodiments, the bar soap further incudes: (c.) a third portion distinct from said first portion and from said second portion, the third portion being a neutral portion absent deodorant and moisturizing formulae, and typically being centrally located and acting as a substrate or support for the other portions. For example, it may be one or more layers, or it may contain recesses to receive the other portions in appropriate areas, such as on opposite sides or opposite ends.

### BRIEF DESCRIPTION OF THE DRAWINGS

The present invention should be more fully understood when the specification herein is taken in conjunction with the drawings appended hereto wherein:

FIG. 1 shows a present invention bar soap having opposite top and bottom portions;

FIG. 2 illustrates another present invention bar soap having opposite ends with distinct moisturizing and deodorant formulae;

FIG. 3 shows a present invention soap with a central base portion;

FIG. 4 shows a present invention soap with plural layers of neutral (base) portions, plural layers of moisturizing formula portions and plural layers of deodorant formula portions;

FIG. 5 shows a present invention soap with a base portion having a separate recess for each of the other portions; and, FIG. 6 illustrates a present invention soap with a base portion having a plurality of recesses for each of the active portions.

### DETAILED DESCRIPTION OF THE PRESENT INVENTION

The present invention is a composite bar of soap formed into a single structure. A portion of the soap is a deodorant soap and a portion of the soap is a gentle moisturizing soap, also sometimes known as a beauty soap. It is two soaps in one for use in cleaning the face and body.

The purpose of said bar of soap is to be able to use it to clean different areas of the body using different portions of the soap. For example, some areas of the body may be dry and/or sensitive and require soap comprised of a gentle moisturizing formula and other areas of the body require soap comprised of a deodorizing formula.

Said bar of soap eliminates the need to use two separate bars of soap, each with a different formula, therefore eliminating the problems associated with using two separate bars of soap. For example, the problem of having two bars of soap toppling over each other and trying to fit them together on the soap dish is eliminated, and there is only one bar needed when travelling.

Said composite bar of soap has a first portion having at least one exposed surface and a second portion having at least one different exposed surface. Said first portion would be comprised of one formula of soap and said second portion would be comprised of a different formula of soap. For example, said first portion of soap is comprised of a deodorant soap formula and said second portion of soap is comprised of a gentle moisturizing soap formula.

Said first portion of soap may contain a perfumed scent and said second portion of soap may contain the same scent as said first portion or anotheror stronger or lighter version of the same or different scent as said first portion or one or both may be completely unscented. For example, said first portion of soap is comprised of a deodorant soap that is scented and said second portion of soap is comprised of a gentle moisturizing soap which is lightly scented.

Said first portion of soap may be comprised of a color and said second portion of soap may be comprised of a contrasting color of said first portion. For example, said first portion of soap may be the color green and said second portion of soap may be the color white. Any color combination would be applicable.

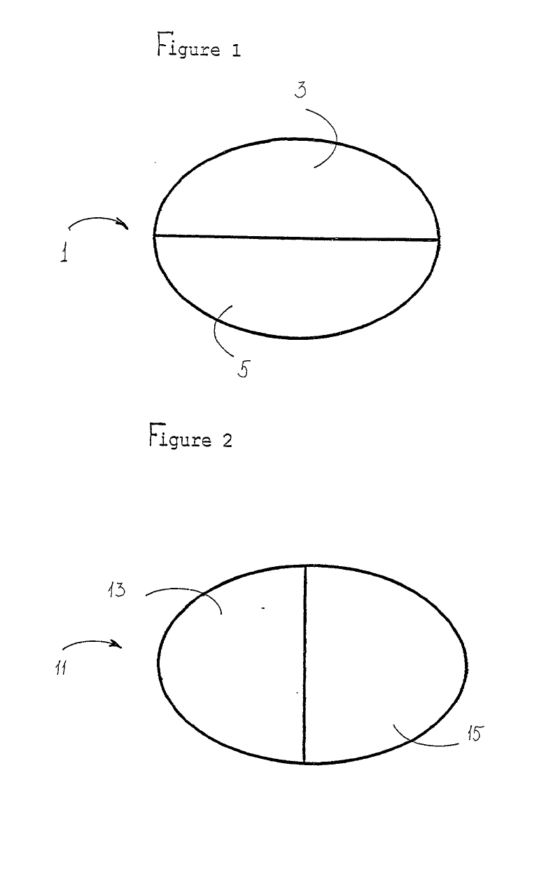

FIG. 1 illustrates the present invention soap bar wherein composite bar of soap 1 includes a top 3 having a deodorant soap formula and a bottom 5 having a moisturizing soap formula.

FIG. 2 shows an alternative embodiment present invention soap bar 11 in its top and bottom view. In this embodiment, the different formulations are side-by-side instead of over-under. Thus, here, left side 13 may be a deodorant soap formula and right side 15 may be a moisturizing soap formula, or vice versa.

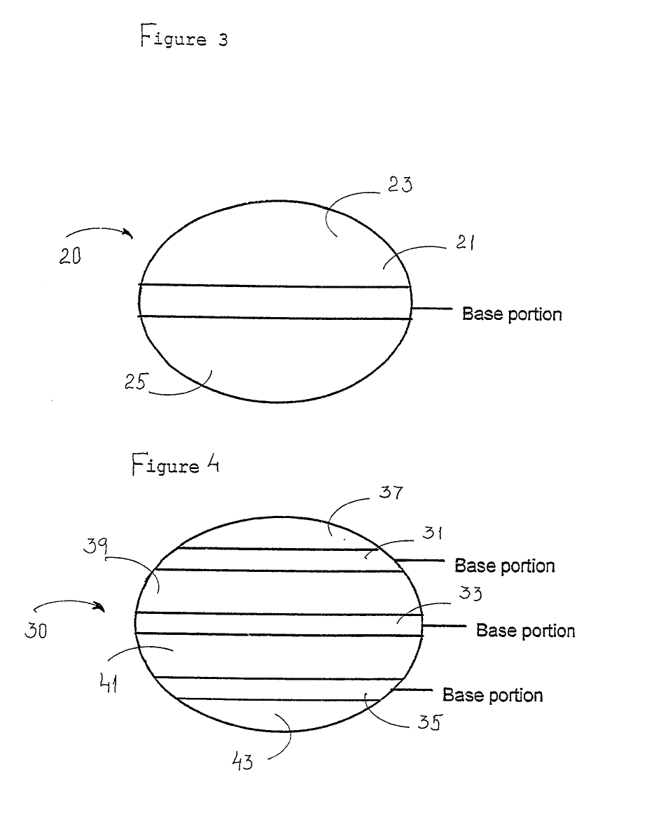

As shown in FIGS. 3 and 4, the composite bar of soap may further include a base portion of soap and said first portion and said second portion may be connected to said base portion in a predetermined pattern. In FIG. 3, there is a neutral wash soap formulation without deodorant or moisturizer as central base portion 21. Atop the base portion 21 is deodorant soap section 23 and below it is moisturizing soap section 25. In FIG. 4, there is a series of base portions 31, 33 and 35, with active soap formulation(s) 37, 39, 41, and 43, as shown. These active soap formulations may be 39 and 41 as deodorant and the other two as moisturizing, for example.

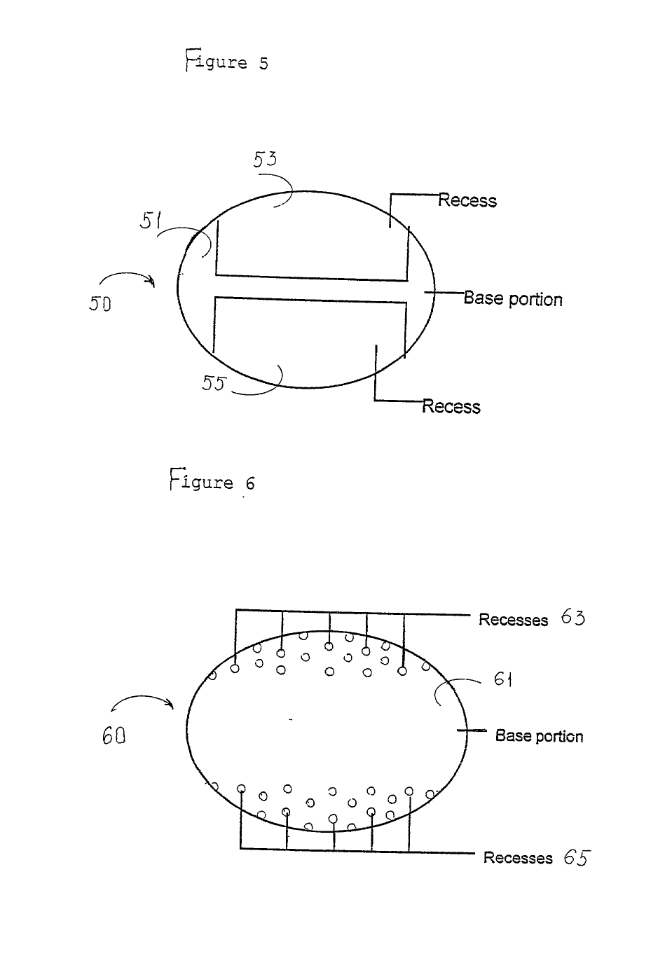

As additional alternatives, the composite bar of soap of the present invention may include a base portion with at least two recesses and said first portion and said second portion are each contained within different of these at least two recesses. FIGS. 5 and 6 illustrate examples thereof. In FIG. 5, the base portion 51 of bar 50 has two recesses with deodorant soap 53 in the upper recess, and moisturizing soap 55 in the lower recess. In FIG. 6, the recesses are deep holes formed by corresponding molds, wherein upper recesses 63 of base portion 61 of bar 60 contain moisturizing soap portions, and lower recesses 65 contain deodorant formulation.

Said bar of soap may also have said first portion and said second portion in a swirling striped patterned configuration, and/or may be in a predetermined shape such as oval, round, square, rectangular, rectangular with rounded edges, etc. In fact, size, color, opacity, density, shape and other characteristics not affecting the functionality of the invention, may vary as desired without exceeding the present invention.

Obviously, numerous modifications and variations of the present invention are possible in light of the above teachings. For example, the soap shown in FIG. 6 could be modified so that it would have recesses on only one side, the central substrate portion would be moisturizing and the recesses on one side would contain deodorant formula. It is, therefore, understood that within the scope of the appended claims, the invention may be practiced otherwise than as specifically described herein.

What is claimed is:

1. A dual function bar soap, which comprises:
a bar soap formed into a single structure, and including:
(a.) a first portion having at least one exposed surface and containing a deodorant soap formula; and,
(b.) a second portion distinct from said first portion and having at least one exposed surface and containing a moisturizing soap formula.

2. The bar soap of claim 1 wherein said first potion and said second portion each form one half of the bar soap.

3. The bar soap of claim 2 wherein said bar soap is an elongated bar soap having a top and a bottom and a first end and a second end, and one of said first portion and said second portion is said top and the other of said first portion and said second portion is said bottom.

4. The bar soap of claim 2 wherein said bar soap is an elongated bar soap having a top and a bottom and a first end and a second end, and one of said first portion and said second portion is said first end and the other of said first portion and said second portion is said second end.

5. The bar soap of claim 1 wherein said bar soap further incudes:
(c.) a third portion distinct from said first portion and from said second portion, said third portion being a neutral portion absent deodorant and moisturizing formulae.

6. The bar soap of claim 5 wherein said third portion is centrally located between said first portion and said second portion.

7. The bar soap of claim 5 wherein said third portion includes a first recess which contains said first portion and a second recess which contains said second portion.

8. The bar soap of claim 5 wherein said third portion includes a first area having a plurality of recesses which contain said first portion and a second area having a plurality of recesses which contain said second portion.

9. The bar soap of claim 5 wherein said third portion is a plurality of separate layers sandwiched between at least two layers of said first portion and at least two layers of said second portion.

10. The bar soap of claim 1 wherein said bar soap has a shape selected from the group consisting of oval, square, round, rectangular and rectangular with rounded corners.
